(19) United States
(12) Patent Application Publication
(10) Pub. No.: US 2025/0213830 A1
(43) Pub. Date: Jul. 3, 2025

(54) BLOOD VESSEL CONNECTION ASSEMBLY

(71) Applicant: Quanzhou Taiyitang Pharmaceutical Co., Ltd., Quanzhou (CN)

(72) Inventor: Renliang ZHENG, Quanzhou (CN)

(21) Appl. No.: 18/985,135

(22) Filed: Dec. 18, 2024

(30) Foreign Application Priority Data
Dec. 28, 2023 (CN) 202311845741.5

(51) Int. Cl. A61M 27/00; A61M 1/36; A61M 39/28

(52) U.S. Cl. CPC A61M 27/002; A61M 1/3659; A61M 39/28; A61M 2027/004; A61M 2202/0413; A61M 2210/12

(57) ABSTRACT

A blood vessel connection assembly is provided in the present application. The blood vessel connection assembly includes two puncture members respectively connected to both ends of a blood vessel, a connection path disposed between the two puncture members and a switch disposed on the connection path and configured for controlling connection and disconnection between the two puncture members; each of the two puncture members includes a needle portion for puncturing, a through channel connected to the needle portion and an operation portion connected to the through channel; the through channel is configured for blood circulation; the needle portion is coaxially sleeved with an abutting member for abutting against an inner wall of the blood vessel; and the through channel is provided with a control assembly for controlling the abutting member to expand or contract.

### CROSS-REFERENCE TO RELATED APPLICATIONS

The present application claims the priority and benefits of Chinese patent application No. 202311845741.5, filed on Dec. 28, 2023. The entirety of Chinese patent application No. 202311845741.5 is hereby incorporated by reference herein and made a part of this specification.

### FIELD OF THE INVENTION

The present application relates to a technical field of medical instruments, in particular to a blood vessel connection assembly.

### BACKGROUND OF THE INVENTION

Rupture of blood vessels, especially rupture of arteries, is prone to damage to body severity if rescue is not in time, and thus emergency hemostasis is required. In order to stop bleeding of a ruptured blood vessel quickly and simply, a common operation in the prior art is to stop bleeding of the blood vessel at a proximal end through compression. However, when a large volume of bleeding is encountered, due to limited compression modes and compression force, a control of the bleeding volume through compression is limited, and it is difficult to achieve effective hemostasis.

Particularly, in some situations with limited conditions or relatively urgent conditions, such as scenes like field first aid or battlefield, although a blood flow passage can be blocked by compression hemostasis, if the blood vessel passage cannot be recovered in time, the blood vessel at a distal end will be insufficient in long-term blood supply. If corresponding parts of a body has long-term ischemia, it is easy to cause limb necrosis and difficult to recover, and there is a case with a high residual rate, and thus further improvement is needed.

### SUMMARY

In order to reduce an occurrence of insufficient blood supply to a blood vessel at the distal end while performing hemostasis on the ruptured blood vessel simply and quickly, the present application provides a blood vessel connection assembly.

The blood vessel connection assembly provided by the present application adopts the following technical solutions.

A blood vessel connection assembly includes two puncture members respectively connected to both ends of a blood vessel, a connection path disposed between the two puncture members and a switch disposed on the connection path and configured for controlling connection and disconnection between the two puncture members; each of the two puncture members includes a needle portion for puncturing, a through channel connected to the needle portion and an operation portion connected to the through channel; the through channel is configured for blood circulation; the needle portion is coaxially sleeved with an abutting member for abutting against an inner wall of the blood vessel; and the through channel is provided with a control assembly for controlling the abutting member to expand or contract.

According to the above technical solution, the needle portion in one of the puncture members is pierced into the blood vessel at the proximal end through the skin through the skin by the handle. The abutting member is expanded through the control assembly and abuts against the inner wall of the blood vessel to intercept blood in the blood vessel. This allows the blood to flow through the needle portion, reducing a possibility of continuing to flow from the ruptured blood vessel. At this time, after the puncture member is successfully connected to the blood vessel at the proximal end through puncturing, a closed switch is opened so that the blood flows to the needle portion in the next puncture member, and thus an air in the connection path between the two puncture members is removed. Thereafter, the above operation is performed on the other puncture member. The needle portion is pierced into the blood vessel at the distal end through the skin, and abuts against the inner wall of the blood vessel through the abutting member. Thus, a blood vessel channel is established at both ends of the ruptured blood vessel to maintain normal blood supply of the blood vessel at the distal end. In this way, a hemostatic effect on the ruptured blood vessel is achieved to facilitate subsequent cleaning and repairing operation.

In some embodiments an outer peripheral wall of the through channel is defined with a plurality of first axial grooves distributed at intervals along an axis of the through channel, and a depth of each of the first axial grooves gradually increases towards a direction away from the needle portion, and the control assembly includes first guide wires slidably provided through the first axial grooves and a pushing member disposed in the through channel for pushing the first guide wires to move towards a direction close to the needle portion, one end of the abutting member away from the needle portion is fixedly connected to the first guide wires, and the pushing member is pushed for driving the first guide wires to slide towards the direction close to the needle portion to expand the abutting member.

According to the above technical solution, after the needle portion penetrates into the blood vessel, the pushing member is pushed to drive the first guide wires and the abutting member which enter the blood vessel along with the needle portion to move towards the direction close to the inner wall of the blood vessel. A size of each of the first guide wires gradually increases towards the direction away from the needle portion as the groove depth of corresponding one of the first axial grooves, so that the first guide wires are moved towards the direction close to the inner wall of the blood vessel and the guiding effect is achieved. Finally, after the abutting member abuts against the inner wall of the blood vessel, the blood in the blood vessel can be intercepted, so that the blood flows into the blood vessel at the distal end through the needle portion. Thus, the occurrence of insufficient blood supply to the blood vessel at the distal end is reduced while hemostasis is performed on the ruptured blood vessel.

In some embodiments, an outer peripheral wall of the through channel is defined with a plurality of second axial grooves distributed at intervals along an axis of the through channel, the control assembly includes a plurality of second guide wires slidably provided through the second axial grooves and a collecting member coaxially sleeved on the through channel to be connected to the plurality of second guide wires; the second guide wires are arc-shaped towards a direction close to the through channel, one end of the abutting member away from the needle portion is fixedly connected to upper ends of the second guide wires, lower ends of the second guide wires are slidably connected to the collecting member, and the collecting member is contracted for pushing the lower ends of the second guide wires to slide towards a direction close to the needle portion, so that the upper ends of the second guide wires move towards a direction close to the inner wall of the blood vessel to expand the abutting member.

According to the above technical solution, it should be noted that in a normal state, a diameter of the collecting member is greater than that of the needle portion and that of the through channel. After the needle portion is pierced into the blood vessel, since the upper ends of the second guide wires are disposed through the second axial grooves and can enter the blood vessel along with the needle portion, the collecting member is contracted to change the diameter of the collecting member. As the diameter is reduced, since the upper ends of the second guide wires are located in the blood vessel, and middle portions of the second guide wires are wrapped by the skin, the middle portions of the second guide wires can be rotated along an abutting point abutting against a skin by the lever principle when the collecting belt is contracted. Thus, the upper ends of the second guide wires move towards the direction close to the inner wall of the blood vessel, and the abutting member is expanded. Finally, after the abutting member abuts against the inner wall of the blood vessel, blood in the blood vessel can be intercepted, so that the blood flows into the blood vessel at the distal end through the needle portion. In this way, the occurrence of insufficient blood supply to the blood vessel at the distal end is reduced while hemostasis is performed on the ruptured blood vessel.

In some embodiments, the pushing member includes a push ring slidably sleeved on the through channel, the plurality of first guide wires are fixedly connected to the push ring, and the through channel is provided with a limiting member for constraining a sliding of the push ring.

According to the above technical solution, after the first guide wires are pushed by the push ring for driving the abutting member to abut against the inner wall of the blood vessel, a path pushed by the push ring is limited through the limiting member to be disposed. Thus, a possibility of retraction of the abutting member caused by pressure impact of blood is reduced, the interception effect on blood in the blood vessel is improved, and the possibility of blood flowing from the ruptured blood vessel is reduced.

In some embodiments, the limiting member includes first clamping teeth provided on an inner wall of the push ring and second clamping teeth provided on an outer peripheral wall of the through channel, the second clamping teeth provided in a plural are arranged at intervals along a length direction of the through channel, the first clamping teeth are engaged with the second clamping teeth, and the first clamping teeth are moveable towards the direction close to the needle portion, when the first clamping teeth are pulled in an opposite direction, a matched latch is formed through crosspiece engagement.

In some embodiments, the limiting member includes a limiting sheet disposed on the inner wall of the push ring, limiting strip grooves into which the limiting sheet to be inserted are formed in the outer peripheral wall of the through channel, and the limiting strip grooves provided in a plural are arranged at intervals along the length direction of the through channel.

In some embodiments, one ends of the first axial grooves are arc-shaped towards the direction close to the needle portion, and upper ends of the first guide wires are arc-shaped towards the direction the needle portion.

In some embodiments, each of the first guide wires includes a sliding section slidably connected to corresponding one of the first axial grooves, a connecting section disposed on the sliding section, and an arc-shaped section disposed on the connecting section, the arc-shaped section is disposed at one end of the connecting section away from the sliding section, and has a free end disposed close to the sliding section, and one end of the abutting member away from the needle portion is fixedly connected to the arc-shaped section and extends to the free end of the arc-shaped section.

In some embodiments, the blood vessel connection assembly further includes a fixing member for fixing the operation portion, the fixing member includes a second binding belt head fixedly connected to the operation portion and a binding belt provided through the second binding belt head, and one end of the binding belt is fixedly connected to the second binding belt head.

In some embodiments, the connection path is provided with a dosing tube.

In summary, the present application has the following beneficial effects.
1. Through an arrangement of two connected puncture members and the switch, blood circulation is controlled through the switch. This hemostasis method is simple to operate, and a blood supply bypass can be quickly established for the hemostasi. An operation time of the hemostasis can be reduced, and the possibility that blood supply is insufficient in blood vessels at the distal end is reduced, so that the possibility of difficult recovery due to limb necrosis caused by insufficient blood supply is reduced.
2. One end of the first axial groove towards the direction close to the needle portion is disposed in an arc shape, and a curvature of the first axial groove gradually increases towards the direction close to the needle portion so as to improve an adaptation range of the puncture members. Further, the upper end of the first guide wire towards the direction close to the needle portion is disposed in an arc shape and can be adapted to an arc direction of the first axial groove. Therefore, a possibility of the first guide wires getting stuck in a sliding process is reduced, and the first guide wire further abuts against the inner wall of the blood vessel after being impacted by blood.

### BRIEF DESCRIPTION OF THE DRAWINGS

FIG. 1 is a schematic view of an overall structure of Embodiment 1 of the present application.

FIG. 2 is a schematic view of a structure of Embodiment 1 of the present application.

FIG. 3 is a partially enlarged view of part A in FIG. 2.

FIG. 4 is a schematic view of a cross-sectional structure of a rubber sleeve being expanded to abut against an inner wall of a blood vessel in Embodiment 1 of the present application.

FIG. 5 is a partially enlarged view of part B in FIG. 4.

FIG. 6 is a schematic view of a structure of a limiting member in Embodiment 1 of the present application.

FIG. 7 is a schematic view of a structure of a dosing tube and a filter in Embodiment 1 of the present application.

FIG. 8 is a schematic view of a structure of a limiting member in Embodiment 2 of the present application.

FIG. 9 is a schematic view of a structure of Embodiment 3 of the present application.

FIG. 10 is a schematic view of a cross-sectional structure of a rubber sleeve being expanded to abut against an inner wall of a blood vessel in Embodiment 3 of the present application.

FIG. 11 is a schematic view of a structure of an alert member in Embodiment 4 of the present application.

FIG. 12 is a schematic view of a cross-sectional structure of the alert member in Embodiment 4 of the present application.

FIG. 13 is a schematic view of a structure of a fixing member in Embodiment 5 of the present application.

FIG. 14 is a schematic view of a structure of an adhesive tape in Embodiment 5 of the present application.

### DETAILED DESCRIPTION

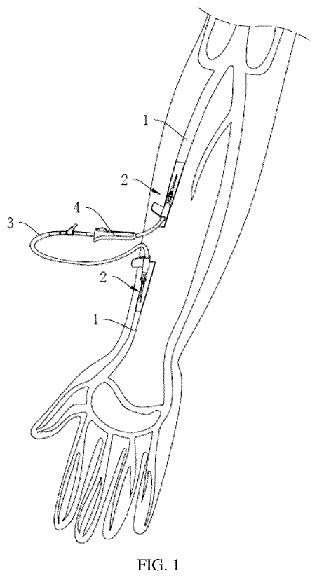

The present application will be further described below in details with reference to FIGS. 1-14.

Embodiments of the present application discloses a blood vessel connection assembly.

### Embodiment 1

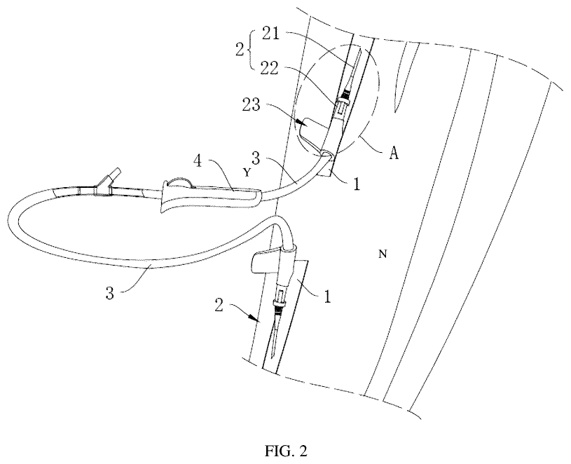

Referring to FIG. 1 and FIG. 2, the blood vessel connection assembly includes two puncture members 2 respectively connected to both ends of a ruptured blood vessel 1, a connection path 3 disposed between the two puncture members 2, and a switch 4 disposed on the connection path 3. In this embodiment, an arterial blood vessel 1 ruptured on an arm is taken as an example.

The connection path 3 is configured to deliver blood, and may be a hose, Specifically, the connection path 3 is an infusion hose in this embodiment. An installation between the puncture members 2 and the infusion hose may be achieved through a quick connector. It should be noted that any pipeline that can be used for conveying liquid can be used as the connection path 3 in this embodiment.

The switch 4 is disposed on the connection path 3 for controlling connection and disconnection between the two puncture members 2. The switch 4 may be a pipeline clamp or a flow regulator. Specifically, the flow regulator adapted to the infusion hose may be selected in this embodiment. It should be noted that any switch 4 that can control the connection and disconnection of the connection path 3 may be used as the switch 4 in this embodiment. The infusion hose and the flow regulator belong to the prior art, and thus are not repeated herein.

Each of the two puncture members 2 specifically includes a needle portion 21 punctured into the blood vessel 1, a through channel 22 connected to the needle portion 21, and an operation portion 23 connected to the through channel 22 and disposed outside the blood vessel 1. It should be noted that a connection between the through channel 22 and the needle portion 21 is relatively smooth, so that an upper end of the through channel 22 can enter the blood vessel 1 together with the needle portion 21.

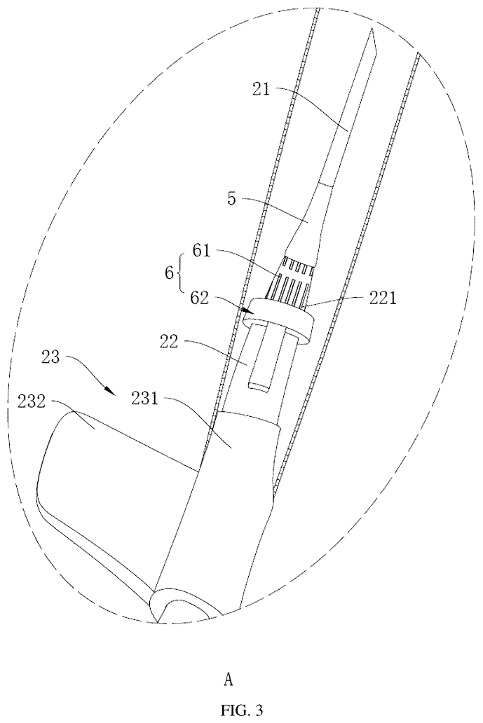

Referring to FIG. 2 and FIG. 3, the through channel 22 is configured for blood circulation, and one end of the operation portion 23 away from the needle portion 21 is connected to the infusion hose. Specifically, the operation portion 23 includes a cannula 231 coaxially and fixedly connected to the through channel 22 and the infusion hose and a handle 232 fixedly connected to an outer peripheral wall of the cannula 231. The handle 232 is pinched to push the needle portion 21 to penetrate into the skin and into the blood vessel 1 so as to connect with the blood in the blood vessel 1.

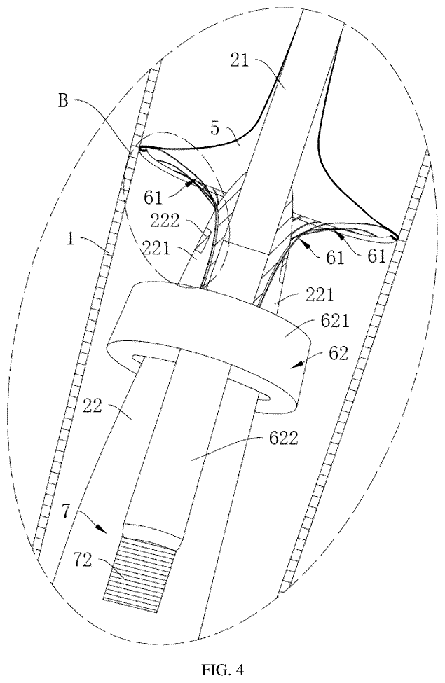

Furthermore, referring to FIG. 3 and FIG. 4, in order to connect the blood at the puncture members 2 on both sides and reduce the possibility that the blood flows from the originally ruptured blood vessel 1, in this embodiment, the needle portion 21 is coaxially sleeved with an abutting member 5 abutting against an inner wall of the blood vessel 1. The through channel 22 is provided with a control assembly 6 for controlling the abutting member 5 to expand or contract. Specifically, the abutting member 5 may be a rubber sleeve, and the control assembly 6 includes first guide wires 61 and a pushing member 62 disposed in the through channel 22 to push the first guide wires 61 to slide along a length direction of the through channel 22. One end of the rubber sleeve away from the needle portion 21 is fixedly connected to the first guide wires 61, and the pushing member 62 pushes the first guide wires 61 to slide towards the direction close to the needle portion 21 to expand the rubber sleeve. After the first guide wires 61 enter the blood vessel 1 together with the needle portion 21 and the rubber sleeve, the rubber sleeve is expanded to abut against the inner wall of the blood vessel 1 to intercept blood in the blood vessel 1. However, when the first guide wires 61 are pushed to slide towards the direction away from the needle portion 21, the rubber sleeve is contracted.

In this embodiment, the first guide wires 61 may be made of materials having relatively good hardness, strength and elasticity such as a polyester material, a polyamide material or an elastic metal material, which is specifically set as required. The outer peripheral wall of the through channel 22 is defined with first axial grooves 221 through which the first guide wires 61 slidably pass. The first axial grooves 221 provided in a plural are distributed at intervals along the axis of the through channel 22. A depth of each of the first axial grooves 221 gradually increases towards the direction away from the needle portion 21. The first guide wires 61 slidably provided through the first axial grooves 221, and a number of the first guide wires 61 corresponds to that of the first axial grooves 221.

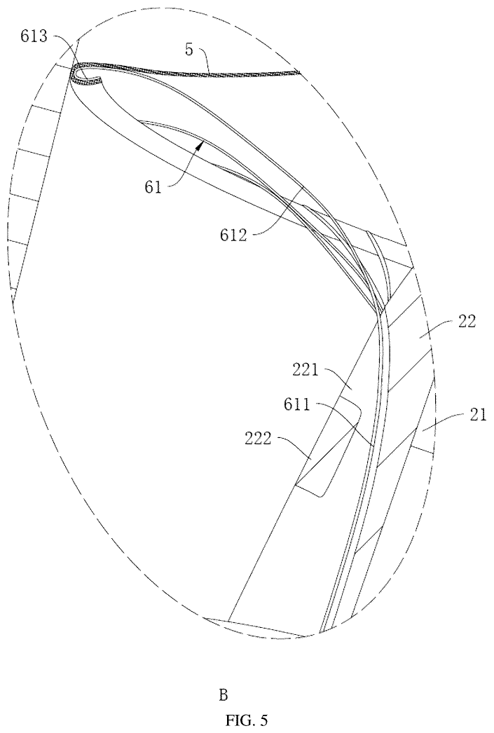

Referring to FIG. 4 and FIG. 5, it should be noted that, in this embodiment, for how to gradually increase the depth of the first axial groove 221 towards the direction away from the needle portion 21, the first axial grooves 221 may be disposed obliquely or in an arc shape. In this embodiment, in order to increase an expanding range of the rubber sleeve driven by the first guide wires 61, one ends of the first axial grooves 221 are arc-shaped towards the direction close to the needle portion 21, and correspondingly, upper ends of the first guide wires 61 are also arc-shaped towards the direction close to the needle portion 21.

It should be noted that, in order to reduce the possibility of the first guide wires 61 slipping away from the first axial grooves 221, in this embodiment, the first axial grooves 221 are provided with limiting blocks 222 protruding from two opposite side walls close to an upper end of the needle portion 21.

Specifically the first guide wire 61 arranged correspondingly includes a sliding section 611 slidably connected to a first axial groove 221, a connecting section 612 disposed on the sliding section 611, and an arc-shaped section 613 disposed on the connecting section 612. In this embodiment, the connecting section 612 is also arc-shaped, specifically disposed in an arc shape towards the direction close to the needle portion 21. The arc-shaped section 613 is disposed at one end of the connecting section 612 away from the sliding section 611, and has a free end disposed close to the sliding section 611. One end of the rubber sleeve away from the needle portion 21 is fixedly connected to the arc-shaped section 613 and extends to the free end of the arc-shaped section 613, so that the possibility that the relatively hard first guide wires 61 abut against the inner wall of the blood vessel 1 to cause damage to the inner wall of the blood vessel 1 is reduced.

It should be noted that, the upper ends of the first guide wires 61 are also arc-shaped towards the direction close to the needle portion 21, which on one hand can be adapted to an arc direction of the first axial grooves 221 to reduce the possibility of the first guide wire 61 getting stuck in the sliding process, and on the other hand, a shape of the rubber sleeve to be expanded can be disposed in an arc-shaped trumpet form. When blood circulation generates an impact force on the first guide wires 61, arc-shaped first guide wires 61 can improve a resistance to blood impact, and reduce the possibility of a gap between the rubber sleeve and the inner wall of the blood vessel 1 after linearly disposed first guide wires 61 are compressed in a straight line, and further contact the inner wall of the blood vessel 1 when subjected to blood impact, thereby improving the tightness between the first guide wire 61 and the inner wall of the blood vessel 1 Further, when the first guide wires 61 are impacted by the blood, the first guide wires 61 may have a reduced compression radian and further abuts against the inner wall of the blood vessel 1. Thus, abutting effect between the first guide wires 61 and the inner wall of the blood vessel 1 is improved. That is, an interception effect on the blood in the blood vessel 1 is improved, and thus a hemostasis effect on the ruptured blood vessel 1 is further improved.

Specifically, the pushing member 62 includes a push ring 621 slidably sleeved on the through channel 22 and a push rod 622 fixedly connected to the push ring 621. The push rod 622 is disposed on an end surface of the push ring 621 away from the needle portion 21. Each of ends of the plurality of first guide wires 61 away from the needle portion 21 is fixedly connected to the push ring 621, and the push ring 621 is disposed between the first axial grooves 221 and the operation portion 23. A force is applied to the push rod 622 towards the direction close to the needle portion 21, so that the first guides wires 61 are pushed to move towards a direction close to the inner wall of the blood vessel 1 along an axial direction of the first axial grooves 221, and the rubber sleeve is expanded and disposed in a trumpet shape. When the rubber sleeve abuts against the inner wall of the blood vessel 1 to intercept the blood in the blood vessel 1, the blood supply deficiency of the blood vessel 1 at the distal end is reduced while the ruptured blood vessel 1 is hemostatic.

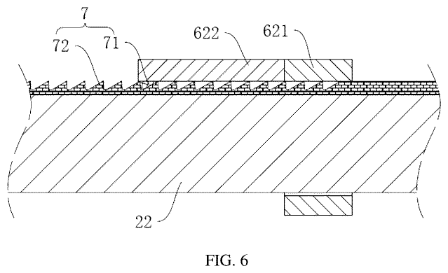

Furthermore, referring to FIG. 4 and FIG. 6, in order to reduce the possibility that the rubber sleeve retracts due to a pressure impact of the blood, in this embodiment, the through channel 22 is provided with a limiting member 7 for constraining a sliding of the push ring 621. The limiting member 7 includes first clamping teeth 71 fixedly connected to an inner wall of the push rod 622 and second clamping teeth 72 fixedly connected to the outer peripheral wall of the through channel 22. The second clamping teeth 72 disposed in a plural are arranged at intervals along the length direction of the through channel 22. The first clamping teeth 71 are engaged with the second clamping teeth 72. The first clamping teeth 71 can easily move towards the direction close to the needle portion 21. However, when the first clamping teeth 71 are pulled in an opposite direction, a matched latch is formed through crosspiece engagement. Thus, a moving length of the push ring 621 is limited; that is, the rubber sleeve is limited after abutting against the inner wall of the blood vessel 1.

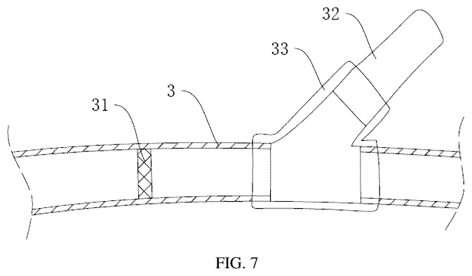

In addition, referring to FIG. 7, in order to reduce the possibility that air enters the blood vessel 1 at the distal end along the infusion hose, in this embodiment, a filter 31, specifically an air filter 31, is disposed on a delivery path of the blood vessel 1 close to the proximal end of the infusion hose. The filter 31 is coaxially and fixedly connected to an inner peripheral wall of the infusion hose. In order to facilitate an operation of injecting liquid medicine or pressurizing injection according to requirements of patients, the infusion hose is provided with a dosing tube 32. Specifically, the infusion hose is fixedly connected with a three-way tube 33, two opposite ends of the three-way tube 33 are fixedly connected to the infusion hose, and the dosing tube 32 is fixedly connected to the three-way tube 33. It should be noted that the dosing tube 32 is disposed at one end of the filter 31 close to the blood vessel 1 at the proximal end, so that the filter 31 can filter substances such as foreign substances, bacteria, air in an injected liquid medicine to prevent them from entering the body of the patient, thereby ensuring a safety and health of the patient.

An implementation principle of the blood vessel connection assembly of this embodiment of the present application is: after the needle portion 21 in one of the puncture members 2 is pierced into the blood vessel 1 near the proximal end through the skin by pinching the handle 232, a force is applied to the push ring 621 in the direction close to the needle portion 21, so that the first guide wires 61 and the rubber sleeve are driven to move towards the direction close to the inner wall of the blood vessel 1. When the rubber sleeve abuts against the inner wall of the blood vessel 1 to intercept the blood in the blood vessel 1, it is necessary to open the closed switch 4, and the blood circulating through the puncture member 2 will exhaust an interior of the infusion hose. It is also possible to observe whether the blood flows normally to determine whether the puncture connection is successful, and then the switch 4 is closed.

Then, the needle portion 21 in the other puncture member 2 is pierced into the blood vessel 1 at the distal end through the skin, and continues to pass through the abutting member 5 to abut against the inner wall of the blood vessel 1, and then the switch 4 is opened to establish a channel for the blood vessel 1. The channel for the blood vessel 1 is established between both ends of the ruptured blood vessel 1, and thus a hemostatic effect on the ruptured blood vessel 1 is achieved. Further, normal blood supply of the blood vessel 1 at the distal end is maintained, so that the possibility that limb necrosis caused by insufficient blood supply and difficult to recover is reduced.

### Embodiment 2

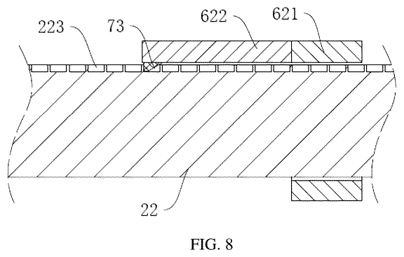

Referring to FIG. 8, differences between this embodiment and Embodiment 1 are that in this embodiment, the limiting member 7 is a limiting sheet 73 fixedly connected to the inner wall of the push ring 621, and the outer peripheral wall of the through channel 22 away from the first axial groove 221 is defined with limiting strip grooves 223 into which the limiting sheet 73 to be inserted. The limiting strip grooves 223 provided in a plural are arranged at intervals along the length direction of the through channel 22. When the push ring 621 is pushed until the rubber sleeve abuts against the inner wall of the blood vessel 1, the limiting sheet 73 is snap-fit into the limiting strip grooves 223, and thus a pushed displacement of the push ring 621 can be limited.

### Embodiment 3

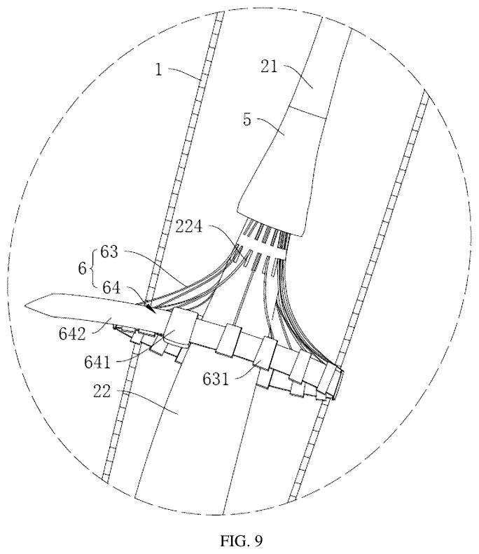

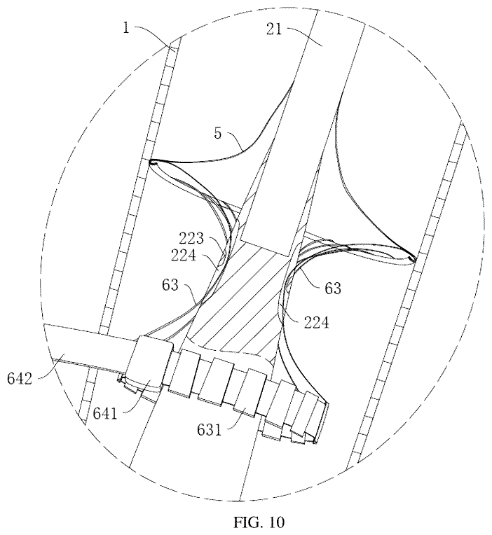

Referring to FIG. 9 and FIG. 10, differences between this embodiment and Embodiment 1 are that in this embodiment, the control assembly 6 specifically includes second guide wires 63 and a collecting member 64 coaxially sleeved on the through channel 22 to connect to the plurality of second guide wires 63. The second guide wires 63 are arc-shaped, specifically arc-shaped towards the direction close to the through channel 22. It should be noted that an arrangement of the second guide wires 63 is similar as that of the first guide wires 61. However, in this embodiment, it is necessary for the second guide wires to be made of a material with a relatively high hardness. One end of the abutting member 5 away from the needle portion 21 is fixedly connected to the upper ends of the second guide wires 63. The outer peripheral wall of the through channel 22 is provided with second axial grooves 224 through which the second guide wires 63 slidably pass. The second axial grooves 224 provided in a plural are distributed at intervals along the axis of the through channel 22, and the second guide wires 63 slidably pass through the second axial grooves 224. A number of the second guide wires 63 corresponds to that of the second axial grooves 224.

It should be noted that, in order to reduce the possibility of the second guide wires 63 slipping away from the second axial grooves 224, in this embodiment, the second axial grooves 224 are provided with limiting blocks 222 protruding from two opposite side walls close to the upper end of the needle portion 21.

The collecting member 64 is disposed as a binding belt specifically including a first binding belt head 641 and a first belt body 92 provided through the first binding belt head 641. One end of the first belt body 92 is fixedly connected to the first binding belt head 641. Lower ends of the second guide wires 63 are provided with sliding sleeves 631, and the sliding sleeves 631 are slidably sleeved on the first belt body 92.

Therefore, in this embodiment, the collecting member 64 may be contracted by pulling the first belt body 92. Since the upper ends of the second guide wires 63 are located in the blood vessel 1, and middle portions of the second guide wires 63 are wrapped by the skin at this time, when the collecting belt is contracted, the lever principle may be used to make the middle portions of the second guide wires 63 rotate along the abutting point abutting against the skin. Thus, the upper ends of the second guide wires 63 move towards the direction close to the inner wall of the blood vessel 1 to expand the rubber sleeve. Finally, after the rubber sleeve abuts against the inner wall of the blood vessel 1, the blood in the blood vessel 1 may be intercepted. Therefore, the blood flows through the needle portion 21 to the blood vessel 1 at the distal end, and the possibility that the blood supply is insufficient in the blood vessel 1 at the distal end is reduced while the ruptured blood vessel 1 is hemostatic.

It should be noted that, in this embodiment, a changed diameter of the collecting member 64 can be limited by using the strap as the collecting member 64, and thus the possibility that the second guide wires 63 retreat after being impacted by blood pressure is reduced.

### Embodiment 4

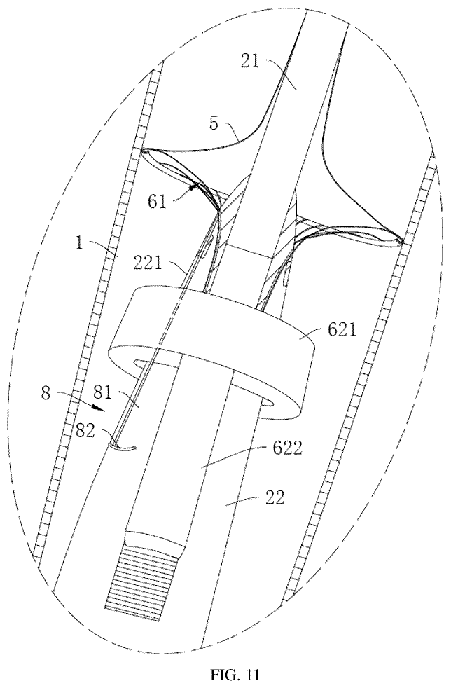

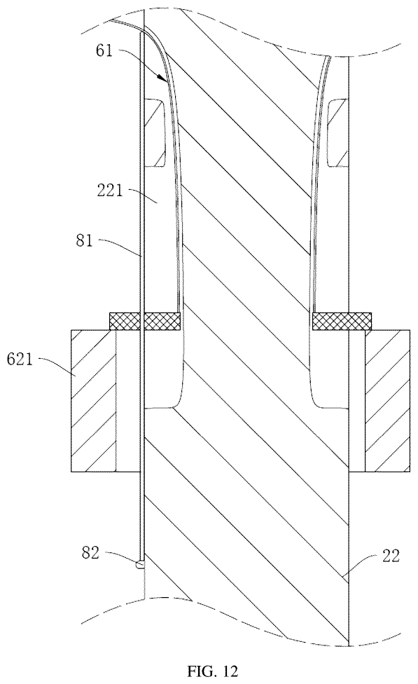

Referring to FIG. 11 and FIG. 12, differences between this embodiment and Embodiment 1 are that in this embodiment, the through channel 22 is provided with an alert member 8 specifically including an alert rod 81 slidably provided through the push ring 621 and an elastic rope 82 fixedly connected to the alert rod 81. An upper end of the alert rod 81 abuts against the first guide wires 61, the alert rod 81 is provided through the push ring 621, and a lower end of the alert rod 81 is fixedly connected to a middle portion of the elastic rope 82. Two ends of the elastic rope 82 are fixedly connected to the outer peripheral wall of the through channel 22.

When the rubber sleeve abuts against the inner wall of the blood vessel 1, a force may be applied on the rubber sleeve and the first guide wires 61 through the pressure impact of the blood. Thus, the first guide wires 61 are slightly deformed. Since the first guide wires 61 are arc-shaped, the first guide wires 61 move towards the direction away from the needle portion 21 when being pressed, so that the alert rod 81 is forced to move towards a direction close to the elastic rope 82. Since the two ends of the elastic rope 82 are fixed to the outer peripheral wall of the through channel 22, the lower end of the alert rod 81 is fixedly connected to the middle of the elastic rope 82, the elastic rope 82 may be pressed to deform. Therefore, an alert effect is achieved, and the possibility that the inner wall of the blood vessel 1 is damaged after the push ring 621 is continuously pushed is reduced.

### Embodiment 5

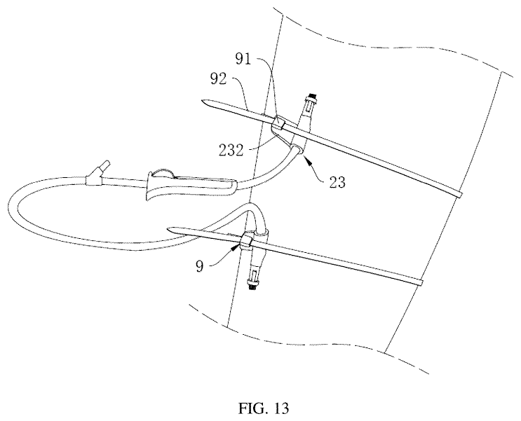

Referring to FIG. 13, differences between this embodiment and Embodiment 1 are that in this embodiment, after the needle portion 21 is pierced into the blood vessel 1, in order to reduce the possibility that the needle portion 21 is detached from the blood vessel 1, a fixing member 9 for fixing the operation portion 23 is further included. It should be noted that the fixing member 9 may also be applied in Embodiment 3.

Specifically, the fixing member 9 includes a second binding belt head 91 fixedly connected to the handle 232 and a second belt body 642 provided through the second binding belt head 91. One end of the second belt body 642 is fixedly connected to the second binding belt head 91. Before providing the second belt body 642 through the second binding belt head 91, it is necessary for the handle 232 to be abutted against an outer skin of the patient. Thereafter, the second belt body 642 is wound around the arm and then passes through the second binding belt head 91. The second belt body 642 is pulled to fix the operation portion 23. Therefore, the possibility that the needle portion 21 is detached from the blood vessel 1 under an influence of external force is reduced.

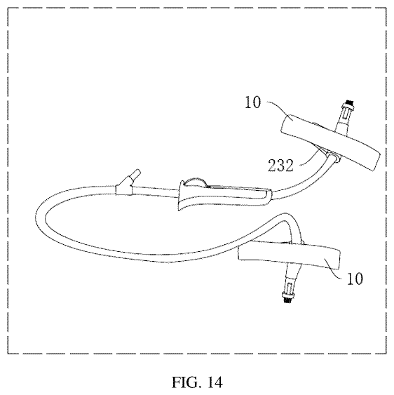

Since the arterial blood vessel 1 on the arm is taken as an example in this embodiment, the second belt body 642 is wound around the arm. However, it should be noted that when hemostasis is performed on the arterial blood vessel 1 at other parts of the body, a length of the second belt body 642 may be correspondingly set. Also, the operation portion 23 may be fixed by a binding belt provided with a hook-and-loop fastener. An adhesive tape 10 may be used for an inconvenient location. A middle portion of the adhesive tape 10 may be adhered to the handle 232, and two ends of the adhesive tape 10 may be adhered to the skin, so that the operation portion 23 may be fixed, as shown in FIG. 14.

The above are all preferred embodiments of the present application and are not intended to limit a protection scope of the present application. Therefore, all equivalent changes made according to a structure, shape and principle of the present application shall fall within the protection scope of the present application.

### LISTING OF REFERENCE SIGNS

1. blood vessel;
2. puncture member;
21. needle portion;
22. through channel;
221. first axial groove;
222. limiting block;
223. limiting strip groove;
224. second axial groove;
23. operation portion;
231. cannula;
232. handle;
3. connection path;
31. filter;
32. dosing tube;
33. three-way tube;
4. switch;
5. abutting member;
6. control assembly;
61. first guide wire;
611. sliding section;
612. connecting section;
613. arc-shaped section;
62. pushing member;
621. push ring;
622. push rod;
63. second guide wire;
631. sliding sleeve;
64. collecting member;
641. first binding belt head;
642. second belt body;
7. limiting member;
71. first clamping teeth;
72. second clamping teeth;
73. limiting sheet;
8. alert member;
81. alert rod;
82. elastic rope;
9. fixing member;
91. second binding belt head;
92. first belt body;
10. adhesive tape.

What is claimed is:

1. A blood vessel connection assembly, comprising: two puncture members respectively connected to both ends of a blood vessel, a connection path disposed between the two puncture members and a switch disposed on the connection path and configured for controlling connection and disconnection between the two puncture members; and
wherein each of the two puncture members comprises a needle portion for puncturing, a through channel connected to the needle portion and an operation portion connected to the through channel; the through channel is configured for blood circulation; the needle portion is coaxially sleeved with an abutting member for abutting against an inner wall of the blood vessel; and the through channel is provided with a control assembly for controlling the abutting member to expand or contract.

2. The blood vessel connection assembly according to claim 1, wherein an outer peripheral wall of the through channel is defined with a plurality of first axial grooves distributed at intervals along an axis of the through channel, and a depth of each of the plurality of first axial grooves gradually increases towards a direction away from the needle portion, and
the control assembly comprises first guide wires slidably provided through the plurality of first axial grooves and a pushing member disposed in the through channel for pushing the first guide wires to move towards a direction close to the needle portion, wherein one end of the abutting member away from the needle portion is fixedly connected to the first guide wires, and the pushing member is pushed for driving the first guide wires to slide towards the direction close to the needle portion to expand the abutting member.

3. The blood vessel connection assembly according to claim 1, wherein an outer peripheral wall of the through channel is defined with a plurality of second axial grooves distributed at intervals along an axis of the through channel,
the control assembly comprises a plurality of second guide wires slidably provided through the plurality of second axial grooves and a collecting member coaxially sleeved on the through channel to be connected to the plurality of second guide wires; wherein the plurality of second guide wires are arc-shaped towards a direction close to the through channel, one end of the abutting member away from the needle portion is fixedly connected to upper ends of the plurality of second guide wires, lower ends of the plurality of second guide wires are slidably connected to the collecting member, and
the collecting member is contracted for pushing the lower ends of the plurality of second guide wires to slide towards a direction close to the needle portion, so that the upper ends of the plurality of second guide wires move towards a direction close to the inner wall of the blood vessel to expand the abutting member.

4. The blood vessel connection assembly according to claim 2, wherein the pushing member comprises a push ring slidably sleeved on the through channel, the first guide wires are fixedly connected to the push ring, and the through channel is provided with a limiting member for constraining a sliding of the push ring.

5. The blood vessel connection assembly according to claim 4, wherein the limiting member comprises first clamping teeth provided on an inner wall of the push ring and second clamping teeth provided on the outer peripheral wall of the through channel, the second clamping teeth are provided in plural and are arranged at intervals along a length direction of the through channel, the first clamping teeth are engaged with the second clamping teeth, the first clamping teeth are moveable towards the direction close to the needle portion, and when the first clamping teeth are pulled in a direction opposite to the direction close to the needle portion, a matched latch is formed through crosspiece engagement.

6. The blood vessel connection assembly according to claim 4, wherein the limiting member comprises a limiting sheet provided on an inner wall of the push ring, limiting strip grooves into which the limiting sheet is to be inserted are formed in the outer peripheral wall of the through channel, and the limiting strip grooves are provided in plural and are arranged at intervals along a length direction of the through channel.

7. The blood vessel connection assembly according to claim 2, wherein first ends of the plurality of first axial grooves are arc-shaped towards the direction close to the needle portion, and upper ends of the first guide wires are arc-shaped towards the direction close to the needle portion.

8. The blood vessel connection assembly according to claim 7, wherein each of the first guide wires comprises: a sliding section slidably connected to a corresponding one of the plurality of first axial grooves, a connecting section disposed on the sliding section, and an arc-shaped section disposed on the connecting section, and
wherein the arc-shaped section is disposed at one end of the connecting section away from the sliding section and the arc-shaped section has a free end disposed close to the sliding section, and one end of the abutting member away from the needle portion is fixedly connected to the arc-shaped section and extends to the free end of the arc-shaped section.

9. The blood vessel connection assembly according to claim 1, further comprising a fixing member for fixing the operation portion, wherein the fixing member comprises a second binding belt head fixedly connected to the operation portion and a binding belt provided through the second binding belt head, and one end of the binding belt is fixedly connected to the second binding belt head.

10. The blood vessel connection assembly according to claim 1, wherein the connection path is provided with a dosing tube.
